(19) United States
(12) Patent Application Publication
(10) Pub. No.: US 2007/0065433 A1
(43) Pub. Date: Mar. 22, 2007

(54) Methods and compositions for the treatment of meconium aspiration syndrome

(76) Inventors: Tom Eirik Mollnes, Bodo (NO); Ola Dirik Saugstad, Olso (NO)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 10/546,253

(22) PCT Filed: Feb. 20, 2004
(86) PCT No.: PCT/US04/05143

§ 371 Date: Aug. 25, 2006

Related U.S. Application Data
(60) Provisional application No. 60/449,045, filed on Feb. 21, 2003.

(51) Int. Cl. A61K 39/395

(52) U.S. Cl. 424/144.1

(57) ABSTRACT

A method for preventing or treating meconium aspiration syndrome (“MAS”) by administering a meconium aspiration syndrome preventing or treating amount of one or more complement inhibitors to a patient likely to develop or suffering from MAS. The complement inhibitors are preferably antibodies that bind to and inhibit complement proteins involved in the formation of the membrane attach complex, preferably anti-Factor D or anti-C5 antibodies. The complement inhibitors can be used alone or in combination with other MAS therapies to decrease the morbidity and mortality caused by MAS.

### CROSS REFERENCE TO RELATED APPLICATIONS

This application claims priority to U.S. Provisional Application Ser. No. 60/449,045, filed Feb. 21, 2003, the disclosure of which is incorporated herein by this reference.

### BACKGROUND OF THE INVENTION

1. Field of the Invention

This invention relates generally to methods and compositions for the prevention and treatment of disease and particularly to methods and compositions for the treatment of meconium aspiration syndrome.

2. Description of the Prior Art

### Immune System—Complement

The immune system protects the body against pathogenic bacteria, viruses, parasites and other harmful organisms. The immune system is divided into two components, the humoral system and the cellular system. Generally, the humoral system includes the complement system and the production of antibodies to defend against pathogens. The complement system, or simply complement, involves the production of proteins that assist the antibodies in the host defense. Complement is a group of at least 30 surface-bound and soluble proteins. The activity of the soluble proteins is destroyed by heating serum at 56° C. for 30 minutes. Complement proteins are involved in the opsonization of microorganisms for phagocytosis, direct killing of microorganisms by lysis, chemotactic attraction of leukocytes to sites of inflammation, activation of leukocytes, and processing of immune complexes.

Complement proteins work in a cascade wherein the binding of one protein promotes the binding of the next protein in the cascade. Activation of the cascade leads to release of biologically active small peptides called anaphylatoxins (C3a, C4a and the most potent C5a) contributing to the inflammatory reaction, and eventually in the formation of a membrane attack complex (C5b-9) that may lyse the target cell. Different complement molecules are synthesized by different cell types, e.g. fibroblasts and intestinal epithelial cells make C1, while most of the components are synthesized in the liver.

The components and mechanism of the complement system are well known. Basically, there are three complement pathways, the classical pathway, the lectin pathway and the alternative pathway. The classical pathway is triggered primarily by immune complexes containing antigen and IgG or IgM, but also by other agents like C-reactive protein. The lectin pathway is triggered by binding of mannose binding lectin (MBL) or ficolins to carbohydrate structures (e.g. mannan) on foreign surfaces. The alternative pathway is activated principally by repeating polysaccharides and other polymeric structures such as those found on bacteria.

The classical pathway is activated when the globular domains of C1q (part of the C1qrs complex) bind to the Fc fragments of IgM or multiple molecules of IgG. In the presence of calcium ions, this binding causes the autocatalytic activation of two C1r molecules. The C1r molecules activate two molecules of C1s. C1s is a serine protease that cleaves C4a from C4b. C4b immediately binds to adjacent proteins or carbohydrates on the surface of the target cell and then binds to C2 in the presence of magnesium ions. C1s cleaves C2b from this complex, yielding the classical pathway C3 convertase, C4b2a. The C3 convertase cleaves many hundreds of molecules of C3 into C3a and C3b. Some molecules of C3b will bind back to C4b2a to yield the classical pathway C5 convertase, C4b2a3b. C5 convertase cleaves C5 into C5a and C5b. C5b binds to the surface of the cell, initiating the formation of the membrane attack complex (MAC). The “lectin pathway” is similar to the classical pathway except it is initiated by the calcium-dependent lectin MBL that binds to terminal mannose groups on the surface of bacteria. MBL is analogous to C1q. When MBL binds to its target, it can interact with two serine proteases known as MASP1 and MASP2 (mannose-binding lectin-associated serine protease), which are analogous to C1r and C1s. The serine proteases cleave C4 into C4b and C4a, and from that point onward, the lectin pathway is identical to the classical pathway.

The alternative complement pathway involves an amplification loop utilizing C3b produced by the classical pathway. Some molecules of C3b generated by the classical pathway C3 convertase are funneled into the alternative pathway. Surface-bound C3b binds Factor B to yield C3bB, which becomes a substrate for Factor D. Factor D is a serine esterase that cleaves the Ba fragment, leaving C3bBb bound to the surface of the target cell. C3bBb is stabilized by properdin (P), forming the complex C3bBbP, which acts as the alternative pathway C3 convertase. As in the classical pathway, the C3 convertase participates in an amplification loop to cleave many C3 molecules, resulting in the deposition of C3b molecules on the target cell. Some of these C3b molecules bind back to C3bBb to form C3bBb3b, the alternative pathway C5 convertase. C5 convertase cleaves C5 into C5a and C5b. C5b binds to the surface of the cell to initiate the formation of the membrane attack complex.

The classical, lectin, and alternative complement pathways all end with the formation of C5 convertase. C5 convertase leads to the assembly of the membrane attack complex (C5b6789n) via the lytic pathway. Components C5-C8 attach to one another in tandem and promote the insertion of one or more monomers of C9 into the lipid bilayer of the target cell. This insertion leads to the formation of pores that cause calcium influx with subsequent cellular activation of nucleated cells or cell lysis and death if the attack is sufficiently strong.

Complement activation has been shown to be a factor in the pathogenesis of several diseases associated with local or systemic inflammation. Kyriakides, et al. demonstrated that the complement alternative pathway plays a significant role in acid aspiration injury (Membrane attack complex of complement and neutrophils mediate the injury of acid aspiration. J. Appl. Physiol. 87(6): 2357-2361, 1999 and Sialyl Lewisx hybridized complement receptor type 1 moderates acid aspiration injury. Am J Physiol Lung Cell Mol Physiol 281: L1494-L1499, 2001). U.S. Pat. No. 6,492,403 discloses a method for treating the symptoms of an acute or chronic disorder mediated by the classical pathway of the complement cascade using furanyl and thienyl amidines and guanidines. U.S. Pat. No. 6,458,360 discloses a soluble recombinant fused protein comprising a polypeptide that contains a recognition site for a target molecule, such as a complement receptor site, and is joined to the N-terminal end of an immunoglobulin chain that is useful for inhibiting complement activation or complement-dependent cellular activation in mammals.

### Meconium Aspiration Syndrome

Meconium is 75% water containing gastric secretions, mucus, bile salts, epithelial cells, free fatty acids, proteins, enzymes, and other products that accumulate in the fetal gastrointestinal tract. Meconium is normally excreted within one or two days after birth. However, stresses such as hypoxia, infections, and other factors during pregnancy can cause the fetus to expel meconium into the surrounding amniotic fluid before and during birth. The presence of meconium in the amniotic fluid is dangerous because the infant may aspirate the meconium into its lungs before or during birth, a condition that may lead to Meconium Aspiration Syndrome (MAS). Meconium is present in the amniotic fluid of approximately 12% (5 to 20%) of all births in the United States. There are approximately 4,000,000 births in the US per year and about 4% of births with meconium stained amniotic fluid will develop MAS, of these at least 5% will die. Therefore, about 20,000 newborn infants develop MAS in the United States per year of these approximately 1000 may die. It is estimated that about 800,000 newborn infants develop MAS worldwide per year.

MAS is associated with a severe form of pneumonia that increases mortality and morbidity rates. Infants born with meconium stained amniotic fluid, especially if they go on developing MAS are at higher risk for developing cerebral palsy and abnormal pulmonary function. Cerebral palsy is permanent for life, currently it is not known if the lung abnormalities persist into adulthood. MAS involves progressive respiratory distress, hypoxia, hypercapnia, and acidosis, thereby necessitating long-term ventilatory treatment in 25-60% of the cases (Wiswell T E Semin Neonatol 2001: 6:225). Hypoxia is caused by a reduction in the oxygen supply to tissues to below physiological levels, despite adequate perfusion of the tissue by the blood, whereas hypercapnia refers to a condition in which there is an excess of carbon dioxide in the blood. Severe cases of MAS require extracorporeal membrane oxygenation (ECMO) for survival. MAS may also result in hypoxemia, vascular shunting, and decreased lung compliance. Meconium aspiration is also known to cause an inflammatory reaction characterized by edema, leukocyte accumulation, and hemorrhage in the lungs. Such reaction usually develops within 2 to 5 hours after the meconium is aspirated into the lungs and may persist for several days. The components of meconium that initiate the inflammatory response and the molecular mechanisms that cause inflammation are not clearly understood. MAS may increase the production of thrombaxone A2 and prostacyclin and may increase the activity of phospholipase A2.

Known methods for treating MAS include surfactant instillation, amnioinfusion, and surfactant lavage. For example, U.S. Pat. No. 5,562,077 discloses a method and apparatus for ventilation and aspiration of meconium, U.S. Pat. No. 6,013,619 discloses pulmonary surfactants and therapeutic uses, including pulmonary lavage, U.S. Pat. No. 5,514,598 discloses methods for the prenatal detection of meconium, and U.S. Pat. No. 6,044,284 discloses an apparatus and method for measuring the concentration of meconium in amniotic fluid. These methods, some of them still considered to be experimental, have met with limited success because they are intrusive or fail to effectively prevent or treat MAS. There is, therefore, a need for new methods and compositions for the prevention and treatment of MAS.

### SUMMARY OF THE INVENTION

It is, therefore, an object of the present invention to provide methods and compositions for preventing and treating meconium aspiration syndrome (“MAS”).

It is another object of the invention to decrease the morbidity and mortality caused by MAS.

These and other objects are achieved using a novel method for preventing or treating MAS that comprises administering a MAS preventing or treating amount of a complement inhibitor to a patient likely to develop or suffering from MAS. The complement inhibitor can be any known complement inhibitor but is preferably an antibody or a functionally equivalent fragment thereof that binds to and inhibits complement proteins in the alternative complement pathway. The antibody or antibody fragment inhibits the action of proteins that are involved in activation of C3 and C5, e.g., Factor D, and inhibits or prevents damage to cells when complement is activated in response to the presence of meconium in a patient.

Other and further objects, features and advantages of the present invention will be readily apparent to those skilled in the art.

### DETAILED DESCRIPTION OF THE INVENTION

### Definitions

The term “patient” means a human or other animal likely to develop or suffering from meconium aspiration syndrome (“MAS”), including bovine, porcine, canine, feline, equine, avian, and ovine animals. Preferably, the patient is a new born human infant.

The term “parenterally” means administration by intravenous, subcutaneous, intramuscular, intratracheal, or intraperitoneal injection.

The term “in conjunction” means that different complement inhibitors are administered to the patient (1) separately at the same or different frequency using the same or different administration routes or (2) together in a pharmaceutically acceptable composition.

The term “concurrently treating” means that complement inhibitors and other therapies are administered to the patient at about the same time, e.g., within about 72 hours before or after administration of complement inhibitors.

The term “functionally equivalent fragments” means antibody fragments that bind to components of the complement system and inhibit complement activation in substantially the same manner as the complete antibody.

This invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise, e.g., reference to “a host cell” includes a plurality of such host cells.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents and publications mentioned herein are incorporated herein by reference to the extent allowed by law for the purpose of describing and disclosing the compounds and methodologies reported therein that might be used with the present invention. However, nothing herein is to be construed as an admission that the invention is not entitled to antedate such disclosure by virtue of prior invention.

### The Invention

In one aspect, the present invention provides a method for preventing and treating meconium aspiration syndrome (“MAS”). The method comprises administering a meconium aspiration syndrome preventing or treating amount of one or more complement inhibitors to a patient. The invention is based upon the discovery that the complement component of the immune system plays a critical role in the development of MAS and that methods and compositions for inhibiting or preventing complement activation can be used to prevent or treat MAS. The methods and compositions are useful for decreasing the morbidity and mortality for patients susceptible to or suffering from MAS.

The complement inhibitors of the present invention are any molecule known to inhibit complement activation in a patient. Generally, the complement inhibitors are small organic molecules, peptides, proteins, antibodies, antibody fragments, or other molecules that function as complement inhibitors. Useful complement inhibitors include compstatin and its functional analogs (inhibits C3), C1 Inhibitor (covalently binds C1r and C1s), C1q inhibitor, C1s inhibitor, sCR1 and its analogues (dissociate all C3 convertases), anti-C5 antibodies (block C5 activation), anti-C5a and anti-C5a receptor antibodies and small-molecule drugs (inhibit C5a signaling pathway), anti-C3a and anti-C3a receptor antibodies and small-molecule drugs (inhibit C3a signaling pathway), anti-C6, 7, 8, or 9 antibodies (inhibit the formation or function of MAC), anti-properdin antibodies (destabilize C3 and C5 convertases in the alternative pathway), and a fusion protein Membrane Cofactor Protein (cofactor for Factor I mediated C3b and C4b cleavage) and Decay Accelerating Factor (DAF) (accelerates decay of all C3 convertases). Other useful inhibitors include C4 bp (accelerates decay of classical pathway C3 convertase (C4b2a)), Factor H (accelerates decay of alternative pathway C3 convertase (C3bBb)), Factor I (proteolytically cleaves and inactivates C4b and C3b (cofactors are required)), Carboxypeptidase N (removes terminal arginine residues from C3a, C4a and C5a), vitronectin (S Protein) and clusterin (binds C5b-7 complex and prevents membrane insertion), and CD59 (inhibits lysis of bystander cells).

Preferably, the complement inhibitors are antibodies or functionally equivalent fragments that bind to and inhibit one or more of the proteins that function in the complement cascade, e.g., C1, C2, C4, C3, C3a, C5, C5a, Factor D, factor B, properdin, MBL or their components, protease cleavage products and receptors. The antibodies bind to a selected complement protein in the complement cascade and inhibit or prevent complement activation when a patient is exposed to meconium. In one embodiment, the complement inhibitor is an anti-C5 antibody or functionally equivalent fragment thereof that binds to C5 and inhibits the formation of C5a and C5b in the complement cascade. The antibody can also be an anti-C5a or anti-C5b antibody that binds to these proteins and inhibits their action in the complement cascade. Most preferably, the complement inhibitor is an anti-Factor D antibody or functionally equivalent fragment thereof that binds to Factor D and inhibits its action in the complement cascade. The antibodies can be a polyclonal or monoclonal antibodies but are preferably monoclonal antibodies.

In the preferred embodiment, the complement inhibitors are compounds that inhibit the alternative or terminal complement pathway. Such inhibitors include anti-Factor D antibodies and their functionally equivalent fragments, anti-properdin antibodies and their functionally equivalent fragments, and anti-C5 antibodies and their functionally equivalent fragments.

Methods for producing antibodies and their functionally equivalent fragments, including polyclonal, monoclonal, monovalent, humanized, human, bispecific, and heteroconjugate antibodies, are well known to skilled artisans.

### Polyclonal Antibodies

Polyclonal antibodies can be produced in a mammal by injecting an immunogen alone or in combination with an adjuvant Typically, the immunogen is injected in the mammal using one or more subcutaneous or intraperitoneal injections. The immunogen may include the polypeptide of interest or a fusion protein comprising the polypeptide and another polypeptide known to be immunogenic in the mammal being immunized. The immunogen may also include cells expressing a recombinant receptor or a DNA expression vector containing the receptor gene. Examples of such immunogenic proteins include, but are not limited to, keyhole limpet hemocyanin, serum albumin, bovine thyroglobulin, and soybean trypsin inhibitor. Examples of adjuvants include, but are not limited to, Freund's complete adjuvant and MPL-TDM adjuvant (monophosphoryl Lipid A, synthetic trehalose dicorynomycolate). The immunization protocol may be selected by one skilled in the art without undue experimentation.

### Monoclonal Antibodies

Monoclonal antibodies can be produced using hybridoma methods such as those described by Kohler and Milstein, Nature, 256:495 (1975). In a hybridoma method, a mouse, hamster, or other appropriate host mammal, is immunized with an immunogen to elicit lymphocytes that produce or are capable of producing antibodies that will specifically bind to the immunogen. Alternatively, the lymphocytes may be immunized in vitro. The immunogen will typically include the polypeptide of interest or a fusion protein containing such polypeptide. Generally, peripheral blood lymphocytes (“PBLs”) cells are used if cells of human origin are desired. Spleen cells or lymph node cells are used if cells of non-human mammalian origin are desired. The lymphocytes are then fused with an immortalized cell line using a suitable fusing agent, e.g., polyethylene glycol, to form a hybridoma cell (Goding, Monoclonal Antibodies: Principles and Practice, pp 59-103 (Academic Press, 1986)). Immortalized cell lines are usually transformed mammalian cells, particularly rodent, bovine, or human myeloma cells. Usually, rat or mouse myeloma cell lines are employed. The hybridoma cells may be cultured in a suitable culture medium that preferably contains one or more substances that inhibit the growth or survival of the unfused immortalized cells. For example, if the parental cells lack the enzyme hypoxanthine guanine phosphonbosyl transferase (HGPRT), the culture medium for the hybridomas typically will include hypoxanthine, aminopterin, and thymidine (HAT medium). The HAT medium prevents the growth of HGPRT deficient cells.

Preferred immortalized cell lines are those that fuse efficiently, support stable high level expression of antibody by the selected antibody producing cells, and are sensitive to a medium such as HAT medium. More preferred immortalized cell lines are murine myeloma lines such as those derived from MOPC-21 and MPC-11 mouse tumors available from the Salk Institute Cell Distribution Center, San Diego, Calif. USA, and SP2/0 or X63-Ag8-653 cells available from the American Type Culture Collection, Rockville, Md. USA. Human myeloma and mouse-human heteromyeloma cell lines also have been described for use in the production of human monoclonal antibodies (Kozbor, J. Immunol. 133:3001 (1984); Brodeur et al., Monoclonal Antibody Production Techniques and Applications, pp. 51-63 (Marcel Dekker, Inc., New York, 1987)). The mouse myeloma cell line NSO may also be used (European Collection of Cell Cultures, Salisbury, Wiltshire UK). Human myeloma and mouse-human heteromyeloma cell lines, well known in the art, can also be used to produce human monoclonal antibodies.

The culture medium used for culturing hybridoma cells can then be assayed for the presence of monoclonal antibodies directed against the polypeptide of interest. Preferably, the binding specificity of monoclonal antibodies produced by the hybridoma cells is determined by immunoprecipitation or by an in vitro binding assay, e.g., radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods. Suitable culture media for this purpose include Dulbecco's Modified Eagle's Medium and RPMI-1640 medium. Alternatively, the hybridoma cells may be grown in vivo as ascites in a mammal.

The monoclonal antibodies secreted by the subclones are isolated or purified from the culture medium or ascites fluid by conventional immunoglobulin purification procedures such as protein A-Sepharose, hydroxylapatite chromatography, gel electrophoresis, dialysis, or affinity chromatography.

The monoclonal antibodies may also be produced by recombinant DNA methods, e.g., those described in U.S. Pat. No. 4,816,567. DNA encoding the monoclonal antibodies of the invention can be readily isolated and sequenced using conventional procedures, e.g., by using oligonucleotide probes that are capable of binding specifically to genes encoding the heavy and light chains of murine antibodies (Innis M. et al. In “PCR Protocols. A Guide to Methods and Applications”, Academic, San Diego, Calif. (1990), Sanger, F. S, et al. Proc. Nat. Acad. Sci. 74:5463-5467 (1977)). The hybridoma cells described herein serve as a preferred source of such DNA. Once isolated, the DNA may be placed into expression vectors. The vectors are then transfected into host cells such as simian COS cells, Chinese hamster ovary (CHO) cells, or myeloma cells that do not otherwise produce immunoglobulin protein. The recombinant host cells are used to produce the desired monoclonal antibodies. The DNA also may be modified, for example, by substituting the coding sequence for human heavy and light chain constant domains in place of the homologous murine sequences or by covalently joining the immunoglobulin coding sequence to all or part of the coding sequence for a non-immunoglobulin polypeptide. Such a non-immunoglobulin polypeptide can be substituted for the constant domains of an antibody or can be substituted for the variable domains of one antigen combining site of an antibody to create a chimeric bivalent antibody.

Monovalent antibodies can be produced using the recombinant expression of an immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking. Similarly, in vitro methods can be used for producing monovalent antibodies. Antibody digestion can be used to produce antibody fragments, preferably Fab fragments, using known methods.

Antibodies and antibody fragments can be produced using antibody phage libraries generated using the techniques described in McCafferty, et al., Nature 348:552-554 (1990). Clackson, et al., Nature 352:624-628 (1991) and Marks, et al., J. Mol. Biol. 222:581-597 (1991) describe the isolation of murine and human antibodies, respectively, using phage libraries. Subsequent publications describe the production of high affinity (nM range) human antibodies by chain shuffling (Marks, et al., Bio/Technology 10:779-783 (1992)), as well as combinatorial infection and in vivo recombination as a strategy for constructing very large phage libraries (Waterhouse, et al., Nuc. Acids. Res. 21:2265-2266 (1993)). Thus, these techniques are viable alternatives to traditional monoclonal antibody hybridoma techniques for isolation of monoclonal antibodies. Also, the DNA may be modified, for example, by substituting the coding sequence for human heavy-chain and light-chain constant domains in place of the homologous murine sequences (U.S. Pat. No. 4,816,567; Morrison, et al., Proc. Nat. Acad. Sci. USA 81:6851 (1984)), or by covalently joining to the immunoglobulin coding sequence all or part of the coding sequence for a non-immunoglobulin polypeptide. Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

Antibodies can also be produced using use electrical fusion rather than chemical fusion to form hybridomas. This technique is well established. Instead of fusion, one can also transform a B-cell to make it immortal using, for example, an Epstein Barr Virus, or a transforming gene “Continuously Proliferating Human Cell Lines Synthesizing Antibody of Predetermined Specificity,” Zurawaki, V. R. et al, in “Monoclonal Antibodies,” ed. by Kennett R. H. et al, Plenum Press, N.Y. 1980, pp 19-33.

### Humanized Antibodies

Humanized antibodies can be produced using the method described by Winter in Jones et al., Nature, 321:522-525 (1986); Riechmann et al., Nature, 332:323-327 (1988); and Verhoeyen et al., Science, 239:1 534-1536 (1988). Humanization is accomplished by substituting rodent CDRs or CDR sequences for the corresponding sequences of a human antibody. Generally, a humanized antibody has one or more amino acids introduced into it from a source that is non-human. Such “humanized” antibodies are chimeric antibodies wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. Humanized forms of non-human (e.g., murine or bovine) antibodies are chimeric immunoglobulins, immunoglobulin chains, or immunoglobulin fragments such as Fv, Fab, Fab′, F(ab′)₂, or other antigen-binding subsequences of antibodies that contain minimal sequence derived from non-human immunoglobulin. Humanized antibodies include human immunoglobulins (recipient antibody) wherein residues from a complementary determining region (CDR) of the recipient are replaced by residues from a CDR of a non-human species (donor antibody) such as mouse, rat, or rabbit having the desired specificity, affinity, and capacity. Sometimes, Fv framework residues of the human immunoglobulin are replaced by corresponding non-human residues. Humanized antibodies also comprise residues that are found neither in the recipient antibody nor in the imported CDR or framework sequences. In general, humanized antibodies comprise substantially all of at least one and typically two variable domains wherein all or substantially all of the CDR regions correspond to those of a non-human immunoglobulin and all or substantially all of the FR regions are those of a human immunoglobulin consensus sequence. Humanized antibodies optimally comprise at least a portion of an immunoglobulin constant region (Fc), typically that of a human immunoglobulin.

### Human Antibodies

Human antibodies can be produced using various techniques known in the art, e.g., phage display libraries as described in Hoogenboom and Winter, J. Mol. Biol., 227:381 (1991) and Marks et al., J. Mol. Biol., 222:581 (1991). Human monoclonal antibodies can be produced using the techniques described in Cole et al., Monoclonal Antibodies and Cancer Therapy, Alan R. Liss, p. 77 (1985) and Boemer et al., J. Immunol., 147(1):86-95 (1991). Alternatively, transgenic animals, e.g., mice, are available which, upon immunization, can produce a full repertoire of human antibodies in the absence of endogenous immunoglobulin production. Such transgenic mice are available from Abgenix, Inc., Fremont, Calif., and Medarex, Inc., Annandale, N.J. It has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA 90:2551 (1993); Jakobovits et al., Nature 362:255-258 (1993); Bruggermann et al., Year in Immunol. 7:33 (1993); and Duchosal et al. Nature 355:258 (1992). Human antibodies can also be derived from phage-display libraries (Hoogenboom et al., J. Mol. Biol. 227:381 (1991); Marks et al., J. Mol. Biol. 222:581-597 (1991); Vaughan, et al., Nature Biotech 14:309 (1996)).

### Bispecific Antibodies

Bispecific antibodies can be produced by the recombinant co-expression of two immunoglobulin heavy-chain/light-chain pairs wherein the two heavy chains have different specificities. Bispecific antibodies are monoclonal, preferably human or humanized, antibodies that have binding specificities for at least two different antigens. In the present invention, one of the binding specificities is for the complement component and the other is for any other antigen, preferably a cell surface receptor or receptor subunit. Because of the random assortment of immunoglobulin heavy and light chains, these hybridomas produce a potential mixture of ten different antibodies. However, only one of these antibodies has the correct bispecific structure. The recovery and purification of the correct molecule is usually accomplished by affinity chromatography.

Antibody variable domains with the desired binding specificities (antibody-antigen combining sites) can be fused to immunoglobulin constant domain sequences. The fusion preferably is with an immunoglobulin heavy chain constant domain comprising at least part of the hinge, CH2, and CH3 regions. Preferably, the first heavy-chain constant region (CH1) containing the site necessary for light-chain binding is present in at least one of the fusions. DNAs encoding the immunoglobulin heavy-chain and, if desired, the immunoglobulin light chain is inserted into separate expression vectors and co-transfected into a suitable host organism. Suitable techniques are shown in for producing bispecific antibodies are described in Suresh et al., Methods in Enzymology, 121:210 (1986).

### Heteroconjugate Antibodies

Heteroconjugate antibodies can be produced using known protein fusion methods, e.g., by coupling the amine group of one an antibody to a thiol group on another antibody or other polypeptide. If required, a thiol group can be introduced using known methods. For example, immunotoxins comprising an antibody or antibody fragment and a polypeptide toxin can be produced using a disulfide exchange reaction or by forming a thioether bond. Examples of suitable reagents for this purpose include iminothiolate and methyl-4-mercaptobutyrimidate. Such antibodies can be used to target immune complement components and to prevent or treat MAS.

The complement inhibitors can be administered to the patient by any means that enables the inhibitor to reach the targeted cells. These methods include, but are not limited to, oral, rectal, nasal, topical, intradermal, subcutaneous, intravenous, intramuscular, intratracehal, and intraperitoneally modes of administration. In one embodiment, the inhibitors are administered by placing the inhibitors directly into the lungs, typically by inhalation or tracheal instillation. Parenteral injections are preferred because they permit precise control of the timing and dosage levels used for administration. For parenteral administration, the complement inhibitors can be, for example, formulated as a solution, suspension, emulsion or lyophilized powder in association with a physiologically acceptable parenteral vehicle. Examples of such vehicles are water, saline, Ringer's solution, dextrose solution, and 5% human serum albumin Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques. For example, a parenteral composition suitable for administration by injection is prepared by dissolving 1.5% by weight of active ingredient in 0.9% sodium chloride solution.

In another aspect, the present invention provides a composition useful for preventing and treating MAS comprising one or more complement inhibitors and one or more pharmaceutically acceptable adjuvants, carriers, excipients, and/or diluents. Acceptable adjuvants, carriers, excipients, and/or diluents for making pharmaceutical compositions are well known to skilled artisans, e.g., Hoover, John E., Remington's Pharmaceutical Sciences, Mack Publishing Co., Easton, Pa. 1975. Another discussion of drug formulations can be found in Liberman, H. A. and Lachman, L., Eds., Pharmaceutical Dosage Forms, Marcel Decker, New York, N.Y., 1980. Most preferably, the inhibitor is mixed with pharmaceutically acceptable carriers to form a composition that allows for easy dosage preparation and administration. Aqueous vehicles prepared from water having no nonvolatile pyrogens, sterile water, and bacteriostatic water and containing at least 0.025M buffer salts, such as sodium phosphate, sodium bicarbonate, sodium citrate, etc. are also suitable to form injectable complement inhibitor solutions. In addition to these buffers, several other aqueous vehicles can be used. These include isotonic injection compositions that can be sterilized such as sodium chloride, Ringer's, dextrose, dextrose and sodium chloride, and lactated Ringer's. Addition of water-miscible solvents, such as methanol, ethanol, or propylene glycol generally increases solubility and stability of the inhibitors in these vehicles. Nonaqueous vehicles such as cottonseed oil, sesame oil, or peanut oil and esters such as isopropyl myristate may also be used as suspension vehicles for the inhibitors. Additionally, various additives which enhance the stability, sterility, and isotonicity of the composition including antimicrobial preservatives, antioxidants, chelating agents, and buffers can be added. Any vehicle, diluent, or additive used would, however, have to be biocompatible and compatible with the inhibitors according to the present invention.

When the complement inhibitor is an antibody or antibody fragment, the formulation is any known formulation suitable for administering antibodies to a patient, e.g., solid antibody formulations such as those disclosed in US Patent Application No. 20020136719, reconstituted lyophilized formulations such as those disclosed in U.S. Pat. No. 6,267,958 or aqueous formulations such as those disclosed in U.S. Pat. No. 6,171,586.

The amount or dosage of complement inhibitor administered to a patient varies depending upon patient type, patient age, patient size, inhibitor type, treatment frequency, administration purpose (therapeutic or prophylactic), and MAS severity. Generally, the complement inhibitors are administered to the patient in dosages of from about 3 to 50 milligrams per kilogram of body weight (mg/kg) per day, preferably from about 5 to 30 mg/kg/day. When administered by inhalation or tracheal instillation, the complement inhibitors are administered to the patient in dosages of from about 0.5-20 mg/kg twice daily. The complement inhibitors can be administered in one dose or the dose can be broken up into smaller doses that can be administered more frequently. The complement inhibitors can be administered alone or in conjunction to combat MAS.

In another aspect, the present invention provides a method for preventing and treating MAS using one or more complement inhibitors in combination with other therapies that reduce the amount of meconium induced into a patient. The method comprises administering one or more complement inhibitors to patient and treating the patient with one or more therapies that reduce the amount of meconium induced into a patient. Using such treatment combinations has advantages such as reducing the dosage of complement inhibitor needed to prevent or treat MAS and increasing the effectiveness of the method that uses complement inhibitors for preventing or treating MAS. Complement inhibitors can be used in combination with aspiration, amnioinfusion, surfactant instillation, surfactant lavage, and other known methods for combating MAS. Such method is useful because it reduces the morbidity and mortality caused by MAS.

### EXAMPLES

This invention can be further illustrated by the following examples of preferred embodiments thereof, although it will be understood that these examples are included merely for purposes of illustration and are not intended to limit the scope of the invention unless otherwise specifically indicated.

### Materials and Methods

### Sera and Reagents

Adult and cord serum were prepared and pooled at 4° C. Adult serum was obtained from healthy persons and cord serum from placenta immediately after delivery (healthy mothers and newborns). Zymosan A (Z-4250) and low molecular weight dextran sulphate (average MW: 5,000; D-7037) were purchased from SIGMA (St. Louis, USA), human serum albumin (200 mg/mL, Vnr. 478172) from Octapharma (Vienna, Austria). C1 inhibitor (Berinert®) was obtained from Aventis (Marburg, Germany).

### Antibodies

Mouse monoclonal antibodies (mAbs) inhibiting C2 (clone 175-62, IgG1) and factor D (clone 166-32, IgG1) and an isotype matched control mAb (clone G3-519, anti HIV 1 gp120, IgG1) were produced by culturing the hybridomas and purifying the monoclonal antibodies by protein A affinity chromatography. Anti-mannose-binding lectin (MBL) (mAb HYB 131-01) was purchased from Antibody Shop (Copenhagen, Denmark).

### Enzyme Immunoassays (EIA)

Complement activation products were measured in EIAs based on mAbs to activation products of the different pathways. C1rs-C1 inhibitor complexes (C1rs-C1inh) classical pathway activation, C4bc both classical and lectin pathway activation, C3bBbP alternative pathway activation, C3bc final common and TCC terminal pathway activation.

C1rs-C1-inhibitor complexes: In a sandwich enzyme immunoassay the wells of certified Maxisorp NUNC-Immunoplates (NUNC A/S, Roskilde, Denmark) were coated with 50 μl of purified mouse mAb Kok 12 at a concentration of 2 μg/mL in 0.1 M carbonate buffer pH 9.6, by overnight incubation at 4° C. Kok 12 reacts with a neoepitope exposed in C1-inhibitor when complexed with a protease. All subsequent incubation steps were performed at 37° C. After each incubation the wells were washed in 200 μl PBS with 0.1% Tweed 20 (SIGMA, St. Louis, Mo.) 3 times. The samples and standards were diluted in PBS containing 0.2% Tween 20 and 10 mM EDTA, tested in triplicates, and incubated for 1 h. After washing, the plates were incubated for another 45 min with a cocktail of goat anti-human C1r (Nordic Immunological Laboratories, Tilburg, The Netherlands) and goat anti-human C1s (Quidel, San Diego, Calif.) diluted in PBS containing 0.2% Tween 20 and 1% dried milk (Molico, Nestle, Vevey, Switzerland). Finally peroxidase-linked anti-goat IgG (Jackson Immuno Research Laboratories, Inc., West Grove, Pa.) was diluted in PBS containing 0.2% Tween 20 and 1% dried milk. Substrate was 0.3 mM 2,2′-azino-di-(3-ethyl)-benzthiazoline sulfonic acid (ABTS, Boehringer Mannheim, Mannheim, Germany) in 0.15 M acetate buffer, pH 4.0 and H₂O₂ to a final concentration of 2.4×10⁻³%. Optical density was determined after 20-30 minutes on a Dynatech MR7000 reader at 405 nm using 490 nm as reference.

C4bc: Maxisorp NUNC-Immunoplates (NUNC A/S, Roskilde, Denmark) were coated with 50 μl of purified mouse mAb C4-1 diluted 1:500 in 0.1 M carbonate buffer pH 9.6, by overnight incubation at 4° C. mAb C4-1 reacts with a neoepitope exposed in activated C4 (C4b and C4c). All subsequent incubation steps were performed at 37° C. After each incubation the wells were washed in 200 μl PBS with 0.1% Tweed 20 (SIGMA, St Louis, Mo.) 3 times. The samples and standards were diluted in PBS containing 0.2% Tween 20 and 10 mM EDTA, tested in triplicates, and incubated for 1 h. After washing, the plates were incubated for another 45 min with biotinylated anti-human C4 (A305; Quidel, San Diego, Calif.) diluted 1/350 in PBS containing 0.2% Tween 20 and 1% dried milk (Molico, Nestle, Vevey, Switzerland). Finally peroxidase-linked streptavidin (Amersham, Buckinghamshire, UK) diluted 1:1000 in PBS containing 0.2% Tween 20 and 1% dried milk. Substrate was 0.3 mM 2,2′-azino-di-3-ethyl)-benzthiazoline sulfonic acid (ABTS, Boehringer Mannheim, Mannheim, Germany) in 0.15 M acetate buffer, pH 4.0 and H₂O₂ to a final concentration of 2.4×10⁻³%. Optical density was determined after 20-30 minutes on a Dynatech MR7000 reader at 405 nm using 490 nm as reference.

C3bBb-properdin complexes (C3bBbP): Activation of the alternative pathway was detected by quantifying the alternative convertase C3bBbP in an EIA. Microtiter plates (Maxisorp, NUNC, Roskilde, Denmark) were incubated at 4° C. overnight with mouse anti-human Factor P, clone # 2 (Quidel, San Diego, Calif.) diluted 1/1000 in 0.05 M carbonate buffer, pH 9.6. Between each further incubation, the plates were washed three times with PBS containing 0.1% (v/v) Tween 20. All incubations were made with 50 μL per well, except for the substrate (100 μL). Standard (see below) was diluted two-fold from 1/100 to 1/3200 and test samples (containing 10 mM EDTA final conc.) were diluted 1/25 in PBS containing 0.2% Tween 20 and 10 mM EDTA. The plates were incubated for 60 min at room temperature. Detection antibody was anti-C3c (Behringwerke A G, Marburg, Germany) diluted 1/1000 in PBS containing 0.2% (v/v) Tween 20. After 45 min incubation at 37° C., horseradish peroxidase-conjugated donkey anti-rabbit Ig (NA9349, Amersham International, Little Chalfont, UK), diluted 1/1000 in PBS containing 0.2% Tween 20, was added. After 45 min incubation at 37° C., substrate was added: ABTS (2,2′-azino-di-(3-ethylbenzthiazoline sulfonic acid), 180 mg/L, diluted in 0.15 M sodium acetate buffer, pH 4.0. H₂O₂ (10 μL of 3%) was added to 12.5 mL substrate solution immediately before use. The standard was a zymosan-activated human serum pool (ZAS) made by incubating serum with 10 mg/mL zymosan (Sigma Chemical Co., St. Louis, Mo.) for 60 min at 37° C. After centrifugation the supernatant was split and stored in aliquots at −70° C. The ZAS was defined to contain 1000 AU/mL of C3bBbP. Optical density was read at 405 nm using 490 nm as reference.

C3bc: Activation of C3 was detected by a sandwich ELISA using the mAb bH6 specific for a common neoepitope on C3b, iC3b and C3c, produced in our own laboratory as capture antibody. Microtiter plates (NUNC Immunoplate II) were obtained from NUNC, Copenhagen, Denmark. mAb bH6 was diluted 1:10,000 in phosphate-buffered saline (PBS) and coating was performed at 4° C. for at least 16 hours. The antigen (standard and samples) was diluted in cold PBS containing 0.2% Tween 20 and 10 mM ethylenediaminetetraacetic acid (EDTA) and incubated at 4° C. for 1 hour. Detection antibody was polyclonal rabbit anti-C3c (Behringwerke A G, Marburg, Germany), diluted 1:10,000 in PBS containing 0.1% Tween 20 and incubated at 37° C. for 45 minutes. Finally peroxidase labelled anti-rabbit Ig (Amersham International, Buckinghamshire, UK), diluted 1:2000 in PBS containing 0.1% Tween 20, was added and incubated at 37° C. for 45 minutes. The plates were washed four times with PBS containing 0.05% Tween in a Dynawasher (Dynatech Laboratories, Alexandria, Va., U.S.A.) between each incubation. Finally substrate was added: ABTS (2,2′-azino-di-(3-ethylbenzthiazoline sulfonic acid) (Boehringer Mannheim, FRG), 180 mg/L, diluted in 0.15 M sodium acetate buffer, pH 4.0: H₂O₂ (10 μL of 3%) was added to 12.5 mL substrate solution immediately before use. Colour formation was measured spectrophotometrically at 405 nm using 490 nm as reference (Dynatech Model MR 7000).

TCC: The soluble terminal SC5b-9 complement complex was detected using a sandwich ELISA based on the mAb aE11 (produced in our own laboratory), specific for a neoepitope exposed in activated C9, as capture antibody. Microtiter plates (NUNC Immunoplate II) were obtained from NUNC, Copenhagen, Denmark. mAb aE11 was diluted 1:10,000 in phosphate-buffered saline (PBS) and coating was performed at 4° C. for at least 16 hours. The antigen (standard and samples) was diluted in cold PBS containing 0.2% Tween 20 and 10 mM ethylenediaminetetraacetic acid (EDTA) and incubated at 4° C. for 1 hour. Detection antibody was biotinylated mAb anti-C6 (clone 9C4; produced in our laboratory), diluted 1:5000 in PBS containing 0.1% Tween 20 and incubated at 37° C. for 45 minutes. The plates were washed with PBS containing 0.05% Tween in a Dynawasher (Dynatech Laboratories, Alexandria, Va., U.S.A.) four times between each incubation. Finally substrate was added: ABTS (2,2′-azino-di-(3-ethylbenzthiazoline sulfonic acid) (Boehringer Mannheim, FRG), 180 mg/L, diluted in 0.15 M sodium acetate buffer, pH 4.0. H₂O₂ (10 μL of 3%) was added to 12.5 mL substrate solution immediately before use. Colour formation was measured spectrophotometrically at 405 nm using 490 nm as reference (Dynatech Model MR 7000).

Preparation of standard for the C1rs-C1 inhibitor and C4bc assays: Activation of a normal human serum pool (NHS) from 10 healthy blood donors, by the classical pathway was made by adding heat aggregated IgG (HAIGG). 10 mg/mL solution of human IgG (Gammaglobulin Kabi, Uppsala, Sweden) in phosphate-buffered saline (PBS) pH 7.2, was incubated in a waterbath at 63° C. for 15 min. The resulting HAIGG-preparation was cooled immediately, and stored at −20° C. One mg of HAIGG per ml prewarmed serumpool was incubated for 30 min. at 37° C. and then centrifuged at 6000×g for 30 min. The supernatant was removed and stored in small aliquots at −70° C. and used as a standard in the assay. The standard was defined to contain 1000 arbitrary units (AU) of C1rs-C1inh and C4bc per mL.

Preparation of standard for the C3bc, C3bBbP, and TCC assays: Alternative pathway activation was induced in normal human serum by incubation for 1 hr at 37° C. with zymosan A (SIGMA, St. Louis, Mo., USA) at a final concentration of 10 mg/mL. The samples were centrifuged at 20,200 G in 1.5 mL Eppendorf tubes in an Eppendorf centrifuge 5417R (Eppendorf-Netheler-Hinz GmbH, Hamburg, Germany) at 4° C. for 30 min. The supernatants were stored at −70° C. and used as a standard in the assay. The standard was defined to contain 1000 arbitrary units (AU) of C3bc, C3bBbP and TCC.

### Meconium

Meconium was obtained and prepared as follows: Individual meconium portions were collected and frozen. After thawing, they were pooled, homogenized, lyophilized, irradiated for sterility, and stored in aliquots at minus 20° C. This preparation was diluted with sterile saline to a final concentration of 135 mg/ml prior to the in vivo experiments. It was further diluted with sterile PBS to a stock concentration of 100 mg/mL prior to the in vitro experiments.

Fractionation of meconium: Meconium was fractionated into a water and a lipid fraction. Bile acids are found in the water fraction and free fatty acids in the lipid fraction. There are unequal amounts of these two fractions in whole meconium. Generally, the weight of lipid fraction was 14% of the original weight of the whole meconium and that of water fraction was 63%. To compare the relative contribution of each fraction, equivalent concentrations of meconium and its fractions were used, implying that a concentration of 14 mg/mL of lipid fraction and 63 mg/mL of water fraction were equivalent to 100 mg/mL of whole meconium.

### In Vitro Experimental Model

Human adult or cord serum was incubated with meconium or equivalent amounts of fractions thereof for one hour at 37° C. Inhibition experiments were performed with pre-incubation of the inhibitors and controls with serum for 5 minutes at 37° C. Then meconium was added and incubated for one hour as described above. In experiments where meconium was mixed with human serum albumin before incubation in serum, pre-incubation was made with different concentration of human serum albumin during different time periods before serum incubation. Complement activation was always stopped at the end of the incubation period by adding ethylenediaminetetraacetic acid (EDTA) to a final conc. of 20 mM.

### In Vivo Piglet Model of Meconium Aspiration Syndrome

Piglets were anesthesized, tracheotomized, and connected to respirator. Then surgery to gain vascular access for infusions, blood samples and invasive monitoring was performed. Lung function variables were registered by a respirator. Fractional inspired oxygen concentration (FiO2) and end-tidal CO2 were continuously monitored separately. After surgery the piglets were first stabilized and then subjected to a hypoxemia period in a gas mixture of 8% O₂ in N₂ until base excess reached −20 mM. The test piglets received meconium (4 mL/kg of a preparation containing 135 mg/mL) via the endotracheal tube and bronchoalveolar lavage was performed after 5 minutes. The control piglet received the same volume of physiologic saline. The piglets were reoxygenated immediately after tracheal installations and observed for 5 hours after reoxygenation. Blood samples were drawn regularly. Oxygenation index (OI) (mean airway pressure×FiO2×100/PaO2) was calculated to assess the

### Example 1

### Induces Complement Activation In Vivo

Meconium aspiration syndrome was induced in new-born piglets using the methods described above. Test animals (n=12) received meconium intratracheally and control animals (n=6) received saline. Observation time was 5 hrs. Complement activation (“TCC”) was measured by using EIA. The results are shown in Table 1. Also, cardio-pulmonary parameters were measured and the disease progress was evaluated by oxygenation index (OI), ventilation index (VI), and pulmonary compliance (PC). Blood samples were obtained after tracheotomy (1), after surgery (2), 20 minutes after hypoxia (3) and then 20 min (4), 60 min (5), 120 min (6), 180 min (7), 240 min (8) and 300 min (9) after reoxygenation. The results are shown in Table 2.

TABLE 1

| | | | TCC | TCC |
|---|---|---|---|---|
| Piglet No. | Sampling Time | Animal Group | AU/mL | Delta-Values |
| 1 | 1 | Control | 1.624 | 0.000 |
| | 3 | | 1.176 | −0.276 |
| | 4 | | 1.580 | −0.027 |
| | 5 | | 1.176 | −0.276 |
| | 6 | | 0.580 | −0.643 |
| | 7 | | 0.788 | −0.515 |
| | 8 | | 0.356 | −0.781 |
| | 9 | | 0.344 | −0.788 |
| 2 | 1 | Control | 1.188 | 0.000 |
| | 2 | | 1.080 | −0.091 |
| | 3 | | 0.900 | −0.242 |
| | 4 | | 0.732 | −0.384 |
| | 5 | | 0.524 | −0.559 |
| | 6 | | 0.408 | −0.657 |
| | 7 | | 0.436 | −0.633 |
| | 8 | | 0.292 | −0.754 |
| | 9 | | 0.224 | −0.811 |
| 4 | 1 | Control | 0.974 | 0.000 |
| | 2 | | 0.727 | −0.254 |
| | 3 | | 0.642 | −0.341 |
| | 4 | | 0.817 | −0.161 |
| | 5 | | 0.976 | 0.002 |
| | 6 | | 0.905 | −0.071 |
| | 7 | | 0.822 | −0.156 |
| | 8 | | 0.514 | −0.472 |
| | 9 | | 0.663 | −0.320 |
| 11 | 1 | Control | 2.192 | 0.000 |
| | 2 | | 2.536 | 0.157 |
| | 3 | | 2.400 | 0.095 |
| | 4 | | 2.504 | 0.142 |
| | 5 | | 1.808 | −0.175 |
| | 6 | | 1.648 | −0.248 |
| | 7 | | 1.228 | −0.440 |
| | 8 | | 1.232 | −0.438 |
| | 9 | | 1.204 | −0.451 |
| 12 | 1 | Control | 0.852 | 0.000 |
| | 2 | | 1.012 | 0.188 |
| | 3 | | 0.596 | −0.300 |
| | 4 | | 0.348 | −0.592 |
| | 5 | | 0.356 | −0.582 |
| | 6 | | 0.360 | −0.577 |
| | 7 | | 0.252 | −0.704 |
| | 8 | | 0.308 | −0.638 |
| | 9 | | 0.264 | −0.690 |
| 21 | 1 | Control | 0.340 | 0.000 |
| | 2 | | 0.288 | −0.153 |
| | 3 | | 0.292 | −0.141 |
| | 4 | | 0.444 | 0.306 |
| | 5 | | 0.340 | 0.000 |
| | 6 | | 0.304 | −0.106 |
| | 7 | | 0.372 | 0.094 |
| | 8 | | 0.236 | −0.306 |
| | 9 | | 0.268 | −0.212 |
| 3 | 1 | Meconium | 0.304 | 0.000 |
| | 2 | (alive) | 0.320 | 0.053 |
| | 3 | | 0.232 | −0.237 |
| | 4 | | 0.524 | 0.724 |
| | 5 | | 0.396 | 0.303 |
| | 6 | | 0.268 | −0.118 |
| | 7 | | 0.248 | −0.184 |
| | 8 | | 0.320 | 0.053 |
| | 9 | | 0.348 | 0.145 |
| 8 | 1 | Meconium | 0.564 | 0.000 |
| | 2 | (alive) | 0.868 | 0.539 |
| | 3 | | 0.344 | −0.390 |
| | 4 | | 0.908 | 0.610 |
| | 5 | | 1.004 | 0.780 |
| | 6 | | 0.640 | 0.135 |
| | 7 | | 0.680 | 0.206 |
| | 8 | | 0.544 | −0.035 |
| | 9 | | 1.204 | 1.135 |
| 9 | 1 | Meconium | 2.728 | 0.000 |
| | 2 | (alive) | 2.232 | −0.182 |
| | 3 | | 1.796 | −0.342 |
| | 4 | | 1.280 | −0.531 |
| | 5 | | 2.028 | −0.257 |
| | 6 | | 1.688 | −0.381 |
| | 7 | | 1.960 | −0.282 |
| | 8 | | 1.788 | −0.345 |
| | 9 | | 1.624 | −0.405 |
| 14 | 1 | Meconium | 0.584 | 0.000 |
| | 2 | (alive) | 0.620 | 0.062 |
| | 3 | | 0.328 | −0.438 |
| | 4 | | 0.516 | −0.116 |
| | 5 | | 0.376 | −0.356 |
| | 6 | | 0.204 | −0.651 |
| | 7 | | 0.356 | −0.390 |
| | 8 | | 0.308 | −0.473 |
| | 9 | | 0.440 | −0.247 |
| 15 | 1 | Meconium | 1.872 | 0.000 |
| | 2 | (alive) | 1.712 | −0.085 |
| | 3 | | 0.932 | −0.502 |
| | 4 | | 1.776 | −0.051 |
| | 5 | | 1.504 | −0.197 |
| | 6 | | 0.888 | −0.526 |
| | 7 | | 1.984 | 0.060 |
| | 8 | | 2.268 | 0.212 |
| | 9 | | 2.608 | 0.393 |
| 16 | 1 | Meconium | 0.440 | 0.000 |
| | 2 | (alive) | 0.276 | −0.373 |
| | 3 | | 0.160 | −0.636 |
| | 4 | | 0.164 | −0.627 |
| | 5 | | 0.180 | −0.591 |
| | 6 | | 0.772 | 0.755 |
| | 7 | | 0.340 | −0.227 |
| | 8 | | 0.716 | 0.627 |
| | 9 | | 0.908 | 1.064 |
| 20 | 1 | Meconium | 0.608 | 0.000 |
| | 2 | (alive) | 0.504 | −0.171 |
| | 3 | | 0.476 | −0.217 |
| | 4 | | 0.476 | −0.217 |
| | 5 | | 0.528 | −0.132 |
| | 6 | | 0.392 | −0.355 |
| | 7 | | 0.324 | −0.467 |
| | 8 | | 0.124 | −0.796 |
| | 9 | | 0.176 | −0.711 |
| 10 | 1 | Meconium | 0.852 | 0.000 |
| | 2 | (dead) | 0.516 | −0.394 |
| | 3 | | 1.080 | 0.268 |
| | 4 | | 0.928 | 0.089 |
| | 5 | | 1.072 | 0.258 |
| | 6 | | 1.264 | 0.484 |
| | 7 | | 1.688 | 0.981 |
| | 8 | | 2.000 | 1.347 |
| | 9 | | | |
| 13 | 1 | Meconium | 0.276 | 0.000 |
| | 2 | (dead) | 0.300 | 0.087 |
| | 3 | | 0.256 | −0.072 |
| | 4 | | 0.348 | 0.261 |
| | 5 | | 0.472 | 0.710 |
| | 6 | | 0.708 | 1.565 |
| | 7 | | 1.384 | 4.014 |
| | 8 | | 1.884 | 5.826 |
| | 9 | | | |
| 17 | 1 | Meconium | 0.280 | 0.000 |
| | 2 | (dead) | 0.200 | −0.286 |
| | 3 | | 0.248 | −0.114 |
| | 4 | | 0.232 | −0.171 |
| | 5 | | 0.832 | 1.971 |
| | 6 | | 1.704 | 5.086 |
| | 7 | | | |
| | 8 | | | |
| | 9 | | | |
| 18 | 1 | Meconium | 0.268 | 0.000 |
| | 2 | (dead) | 0.208 | −0.224 |
| | 3 | | 0.128 | −0.522 |
| | 4 | | 0.152 | −0.433 |
| | 5 | | 0.188 | −0.299 |
| | 6 | | 0.240 | −0.104 |
| | 7 | | 0.592 | 1.209 |
| | 8 | | | |
| | 9 | | | |
| 22 | 1 | Meconium | 0.292 | 0.000 |
| | 2 | (dead) | 0.404 | 0.384 |
| | 3 | | 0.188 | −0.356 |
| | 4 | | 1.316 | 3.507 |
| | 5 | | 4.568 | 14.644 |
| | 6 | | 3.820 | 12.082 |
| | 7 | | | |
| | 8 | | | |
| | 9 | | | |

TABLE 2

| | R 60 | R 120 | R 180 | R 240 | R 300 |
|---|---|---|---|---|---|
| | 1 hr | 2 hrs | 3 hrs | 4 hrs | 5 hrs |
| TCC vs OI | r = 0.67 | r = 0.61 | r = 0.81 | r = 0.71 | r = 0.56 |
| | p = 0.040 | p = 0.012 | p < 0.0005 | p = 0.006 | p = 0.057 |
| TCC vs VI | r = 0.84 | r = 0.78 | r = 0.82 | r = 0.85 | r = 0.74 |
| | p < 0.0005 | p < 0.0005 | p < 0.0005 | p < 0.0005 | p = 0.006 |
| TCC vs PC | r = −0.132 | r = −0.233 | r = −0.370 | r = −0.632 | r = −0.692 |
| | p = 0.61 | p = 0.386 | p = 0.194 | p = 0.020 | p = 0.013 |

Referring to Table 1, plasma TCC increased significantly in the meconium group compared with the controls. TCC increased significantly more in the meconium animals with fatal outcome than in the survivors (p<0.0005). Referring to Table 2, there was a close and highly significant correlation between TCC and OI and VI during the whole observation period and between TCC and PC at the end of the observation period (changes in pulmonary compliance occur late compared with OI and VI). The results show that (1) complement is activated systemically in MAS, (2) TCC is markedly and significantly higher in animals with fatal outcome during the observation period than in those surviving, (3) TCC is closely correlated with morbidity as measured by oxygenation index, ventilation index, and pulmonary compliance, and (4) TCC is an important trigger of the systemic inflammation and clinical outcome in MAS. There is, therefore, no doubt that TCC is correlated with morbidity and mortality in MAS.

### Example 2

Meconium was incubated in human umbilical cord serum for 60 min at 37° C. and TCC was measured by detecting any increase in the soluble terminal SC5b-9 complement complex in a dose-response manner. The results for three experiments are shown in Table 3.

TABLE 3
TCC (AU/mL) Formation in Serum Incubated With Meconium
(Upper Part; Three Experiments) Compared with Human
Serum Albumin (HSA) as Control in Two of the Experiments
(Lower Part) For 60 Minutes

| Meconium | TCC* | TCC | TCC |
|---|---|---|---|
| mg/mL | (Exp. 1) | (Exp. 2) | (Exp. 3) |
| 10 | 73.3 | 117 | 91.0 |
| 5 | 50.8 | 70.4 | 57.1 |
| 2.5 | 30.7 | 48.3 | 47.2 |
| 1.25 | 24.8 | 38.2 | 30.6 |
| 0.625 | 17.5 | 25.5 | 25.1 |
| 0.313 | 11.5 | 3.7 | 18.7 |
| 0.156 | 10.2 | 5.9 | 15.1 |
| 0 | 5.5 | 4.8 | 8.0 |
| Baseline | 2.4 | 2.5 | 3.5 |

| HSA | TCC | TCC |
|---|---|---|
| mg/mL | (Exp 1) | (Exp 2) |
| 10 | 3.54 | 9.96 |
| 5 | 4 | 8.14 |
| 2.5 | 4.45 | 6.66 |
| 1.25 | 4.07 | 5.62 |
| 0.625 | 4.11 | 5.64 |
| 0.313 | 3.84 | 5.33 |
| 0.156 | 4.1 | 5.05 |

* TCC in AU/mL

Referring to Table 3, the results show that meconium activated complement substantially in contrast to human serum albumin (HSA) and that HSA had no effect on complement activation. Therefore, meconium induces complement activation in umbilical cord serum.

### Example 3

Since newborns have lower concentrations of complement components than adults, human umbilical cord serum and adult serum were compared with respect to the ability of meconium and zymosan (a well-known potent complement activator) to form TCC. The results are shown in Table 4.

TABLE 4

| Conc. mg/mL | Meconium-Cord (TCC)* | Meconium-Adult (TCC) |
|---|---|---|
| 10 | 91.0 | 140.9 |
| 5 | 57.1 | 128.7 |
| 2.5 | 47.2 | 106.3 |
| 1.25 | 30.6 | 97.5 |
| 0.625 | 25.1 | 99.6 |
| 0.313 | 18.7 | 94.2 |
| 0.156 | 15.1 | 85.3 |
| 0 | 8.2 | 37.4 |
| | Zymosan-Cord (TCC) | Zymosan-Adult (TCC) |
| 1 | 214.2 | 943.5 |
| 0.1 | 123.8 | 228.1 |
| 0.05 | 89.1 | 132.2 |
| 0.025 | 56.7 | 102.3 |
| 0.0125 | 37.4 | 71.8 |
| 0.0062 | 24.7 | 67.5 |
| 0.0031 | 17.7 | 46.1 |
| 0 | 8.2 | 37.4 |
| Baseline | 3.3 | 2.6 |

*TCC in AU/mL

Referring to Table 4, the maximum level of TCC formed by zymosan in umbilical cord serum was approximately 25% of that achieved in adult serum, a result consistent with the lower amount of complement components in the former. Similarly, meconium activated complement to a greater extent in adult than in umbilical cord serum, although a substantial activation was seen in the latter. One representative of three experiments is shown. The subsequent experiments were performed with umbilical cord serum since this is the pathophysiological relevant medium for meconium aspiration syndrome.

### Example 4

Human meconium was fractionated into the lipid fraction (containing e.g. free fatty acids) and the water fraction (containing e.g. bile acids) and the effects of the fractions on TCC formation was examined by incubation in human serum for 60 min at 37° C. The results are shown in Table 5.

TABLE 5

| | Whole | Lipid | Water | Lipid + | |
|---|---|---|---|---|---|
| Conc. | meconium | fraction | fraction | Water | |
| mg/mL | TCC* | TCC | TCC | TCC | Controls |
| 10 | 99.2 | 63.9 | 77.2 | 128.0 | |
| 5 | 61.7 | 41.4 | 35.8 | 57.1 | |
| 2.5 | 39.5 | 26.3 | 27.6 | 39.7 | |
| 1.25 | 28.7 | 24.8 | 19.5 | 25.1 | |
| 0.625 | 23.5 | 18.6 | 16.5 | 18.5 | |
| 0.313 | 18.3 | 15.5 | 13.7 | 16.1 | |
| 0.156 | 15.1 | 12.8 | 12.2 | 13.0 | |
| 0 | | | | | 9.1 |
| Baseline | | | | | 3.7 |

*TCC in AU/mL

Referring to Table 5, whole meconium and equivalent concentrations of the fractions as well as reconstituted fractions showed that the lipid and the water fraction contributed equally to the TCC formation and that mixing the fractions restored the activity of whole meconium. One representative of three experiments is shown.

### Example 5

The capacity of albumin to bind free fatty acids has been postulated as a possible therapeutic approach for meconium aspiration syndrome by instillation albumin into the lungs. The effect of pre-incubating meconium with human albumin on serum complement activation capacity was investigated. Increasing concentrations of albumin was added to meconium 15 min before incubation in serum for 60 min at 37° C. The results are shown in Table 6. Median and range of three separate experiments are shown.

TABLE 6

| HSA | TCC* | TCC | TCC |
|---|---|---|---|
| mg/mL | (Exp 1) | (Exp 2) | (Exp 3) |
| 20.00 | 85.5 | 64.8 | 54.0 |
| 10.00 | 91.0 | 69.7 | 57.4 |
| 5.00 | 84.4 | 71.8 | 57.4 |
| 2.50 | 88.7 | 74.4 | 61.8 |
| 1.25 | 92.9 | 65.0 | 57.7 |
| 0.00 | 105.2 | 79.3 | 60.4 |
| No meconium | 7.5 | 7.0 | 5.2 |
| Baseline | 2.8 | 4.0 | 0.2 |

*TCC in AU/mL

Referring to Table 6, the results show that albumin did not inhibit TCC formation.

### Example 6

The role of C1-inhibitor as an inhibitor of the classical and lectin pathway of complement system is well known. Recently, it was also described an inhibitory effect of C1-inhibitor on the alternative pathway as well. The effect of C1-inhibitor on meconium-induced complement activation was therefore investigated using the techniques described above. Low molecular weight dextran sulphate is a well-known potentiator of C1-inhibitor function. Meconium was incubated in serum in the presence of C1 inhibitor, dextran or HSA. The results are shown in Table 7. One representative of three experiments is shown.

TABLE 7

| | TCC* |
|---|---|
| Dextran mg/mL | |
| 20 | 0.05 |
| 10 | 0.06 |
| 5 | 0.10 |
| 2.5 | 0.16 |
| 1.25 | 0.26 |
| 0 | 1.49 |
| C1-inhibitor U/mL | |
| 4 | 1.13 |
| 2 | 1.22 |
| 1 | 1.59 |
| 0.5 | 1.47 |
| 0.25 | 1.54 |
| 0 | 1.53 |
| HSA mg/mL | |
| 6.4 | 1.42 |
| 3.2 | 1.62 |
| 1.6 | 1.55 |
| 0.8 | 1.61 |
| 0.4 | 1.78 |
| 0 | 1.72 |

*TCC in AU/mL

Referring to Table 7, only a modest decrease in TCC formation was observed for C1-inhibitor concentrations up to four times the physiological concentration (4 U/mL). Low molecular weight dextran abolished TCC formation dose-dependently, consistent with a very limited effect of exogenously added C1-inhibitor. The dextran sulphate effect could be due to potentiation of endogenous C1-inhibitor present in serum or an effect on other complement components, including alternative pathway factor H. Human serum albumin was used as a control and had no inhibitory effect on complement activation. Thus, meconium-induced complement activation is modestly inhibited by C1-inhibitor and abolished by low molecular weight dextran sulphate.

### Example 7

Meconium and its lipid and water fractions were incubated in human umbilical cord serum for 60 min at 37° C. and activation products form the classical (C1rs-C1-inhibitor complexes), classical and lectin (C4bc) and alternative (C3bBbP) pathways were measured. The results on unfractionated meconium are shown in Table 8 and presented as times increase from baseline.

TABLE 8

| Meconium | Exp. 1 | Exp. 2 | Exp. 3 |
|---|---|---|---|
| Conc. mg/mL | C3bBbP | C3bBbP | C3bBbP |
| 10 | 3.29 | 2.50 | 2.64 |
| 5 | 2.58 | 1.58 | 2.37 |
| 2.5 | 2.44 | 1.71 | 1.78 |
| 1.25 | 2.11 | 1.31 | 2.17 |
| 0.625 | 1.83 | 1.65 | 1.49 |
| 0.313 | 1.86 | 1.16 | 2.40 |
| 0.156 | 1.58 | 1.16 | 1.62 |
| 0 | 1 | 1 | 1 |
| | C4bc | C4bc | C4bc |
| 10 | 1.58 | 1.67 | 1.10 |
| 5 | 1.15 | 1.05 | 1.10 |
| 2.5 | 1.19 | 1.43 | 0.79 |
| 1.25 | 1.18 | 0.95 | 0.87 |
| 0.625 | 1.19 | 1.38 | 0.87 |
| 0.313 | 1.06 | 0.95 | 1.06 |
| 0.156 | 0.91 | 1.14 | 0.94 |
| 0 | 1 | 1 | 1 |
| | C1rs-C1inh | C1rs-C1inh | C1rs-C1inh |
| 10 | 0.88 | 0.72 | 0.63 |
| 5 | 0.68 | 1.03 | 0.74 |
| 2.5 | 0.96 | 0.78 | 0.70 |
| 1.25 | 0.69 | 1.02 | 0.79 |
| 0.625 | 0.95 | 0.72 | 0.72 |
| 0.313 | 0.75 | 0.97 | 0.77 |
| 0.156 | 0.95 | 0.78 | 0.92 |
| 0 | 1 | 1 | 1 |

Referring to Table 8, meconium induced a marked increase in C3bBbP, but not in C1rs-C1inh complexes or C4bc, indicating an alternative pathway mechanism of activation. Median and range of three separate experiments is shown and times increase from baseline is indicated. Both the lipid and the water fractions showed the same pattern of activation as whole meconium. Thus, meconium activates the complement system via the alternative pathway.

### Example 8

Meconium was incubated in human umbilical cord serum for 60 min at 37° C. in the presence of inhibitory monoclonal antibodies to C2 (classical/lectin pathway), mannose binding lectin (MBL, lectin pathway), factor D (alternative pathway) or the isotype control antibody G3-519 (all antibodies of IgG1 subclass). The results of TCC formation are shown in Table 9.

TABLE 9

| Ab mg/mL | Anti-Factor D | Isotype ctr. | Anti-C2 | Anti-MBL |
|---|---|---|---|---|
| 25 | 16.0 | 108.5 | 101.7 | 99.9 |
| 12.5 | 19.2 | 114.4 | 101.5 | 107.6 |
| 6.25 | 95.5 | 106.6 | 108.9 | 116.1 |
| 3.13 | 107.9 | 117.7 | 112.7 | 101.9 |

Baseline: 9.7

Referring to Table 9, anti-factor D completely prevented TCC formation, whereas the other antibodies had no effect.

### Example 9

Using the in vivo model described herein, two piglets received meconium (P1 and P2) and one control received saline. The results are shown in Table 10.

TABLE 10

| Time point | TCC, P1 | TCC, P2 | TCC, Control |
|---|---|---|---|
| 2 | 0.13 | 0.3 | 0.3 |
| 3 | 0.3 | 0.9 | 0.4 |
| 4 | 0.3 | 2.9 | |
| 5 | | 2.5 | 0.3 |
| 6 | | 2.1 | 0.3 |
| 7 | 1.0 | 2.4 | 0.4 |
| 8 | 0.6 | | 0.2 |
| 9 | 1.5 | | 0.3 |
| | OI, P1 | OI, P2 | OI, C |
| 2 | 1.46 | 1.48 | 0.98 |
| 3 | 2.82 | 4.12 | 1.6 |
| 4 | 1.44 | 6.7 | |
| 5 | 5.05 | 14.23 | 1.4 |
| 6 | 9.3 | 15.84 | 1.3 |
| 7 | 28.93 | 22.26 | 1.6 |
| 8 | 29.54 | | 1.2 |
| 9 | | | 1.4 |

Referring to Table 10, oxygenation index and TCC increased in both test piglets but not in the control. Test piglet P2 had a rapid increase in both oxygenation index and TCC and died after 3 hrs, whereas the other test piglet died immediately before end of the experiment (5 hrs), and had a later and less pronounced increase in TCC. These results show that complement inhibitors can be used to prevent or treat MAS.

In the specification, there have been disclosed typical preferred embodiments of the invention and, although specific terms are employed, they are used in a generic and descriptive sense only and not for purposes of limitation, the scope of the invention being set forth in the following claims. Obviously many modifications and variation of the present invention are possible in light of the above teachings. It is therefore to be understood that within the scope of the appended claims the invention may be practiced otherwise than as specifically described.

What is claimed is:

1. A method for preventing or treating meconium aspiration syndrome comprising administering a meconium aspiration syndrome preventing or treating amount of one or more complement inhibitors to a patient likely to develop or suffering from meconium aspiration syndrome.

2. The method of claim 1 wherein the complement inhibitor is selected from the group consisting of compstatin and its functional analogs, C1 Inhibitor, C1q inhibitor, C1s inhibitor, sCR1 and its analogues, anti-C5 antibodies and their functionally equivalent fragments, anti-C5a antibodies and their functionally equivalent fragments, anti-C5a receptor antibodies and their functionally equivalent fragments, anti-C3a antibodies and their functionally equivalent fragments, anti-C3a receptor antibodies and their functionally equivalent fragments, anti-C6 antibodies and their functionally equivalent fragments, anti-C7 antibodies and their functionally equivalent fragments, anti-C8 antibodies and their functionally equivalent fragments, anti-C9 antibodies and their functionally equivalent fragments, anti-properdin antibodies and their functionally equivalent fragments, fusion protein Membrane Cofactor Protein, Decay Accelerating Factor (DAF), C4 bp, Factor H, Factor I, Carboxypeptidase N, vitronectin (S Protein), clusterin, and CD59.

3. The method of claim 1 wherein the complement inhibitor is an antibody or a functionally equivalent fragment thereof.

4. The method of claim 3 wherein the antibody is an anti-Factor D antibody.

5. The method of claim 3 wherein the antibody is selected from the group consisting of anti-properdin antibodies and functionally equivalent fragments thereof, an anti-C5 antibodies and functionally equivalent fragments thereof, and anti-C5a antibodies and functionally equivalent fragments thereof.

6. The method of claim 1 wherein the complement inhibitor inhibits a component of the alternative complement pathway.

7. The method of claim 1 wherein the complement inhibitor is administered to the patient in dosages of from about 3 to 50 milligrams per kilogram of body weight per day.

8. The method of claim 1 wherein the complement inhibitor is administered into the lungs of the patient.

9. The method of claim 1 wherein the complement inhibitor is administered into the lungs of the patient using a method selected from the group consisting of inhalation and tracheal instillation.

10. A composition useful for the prevention and treatment of meconium aspiration syndrome comprising one or more complement inhibitors and one or more pharmaceutically acceptable adjuvants, carriers, excipients, and diluents.

11. The composition of claim 10 comprising two or more complement inhibitors.

12. A method for preventing or treating meconium aspiration syndrome comprising;
administering a meconium aspiration syndrome preventing or treating amount of one or more complement inhibitors to a patient likely to develop or suffering from meconium aspiration syndrome, and
concurrently treating the patient with one or more conventional therapies that reduce the amount of meconium induced into the patient.

13. The method of claim 11 wherein the conventional therapy is selected from the group consisting of aspiration, amnioinfusion, surfactant instillation, and surfactant lavage.

14. The method of claim 11 wherein the complement inhibitor is selected from the group consisting of compstatin and its functional analogs, C1 Inhibitor, C1q inhibitor, C1s inhibitor, sCR1 and its analogues, anti-C5 antibodies and their functionally equivalent fragments, anti-C5a antibodies and their functionally equivalent fragments, anti-C5a receptor antibodies and their functionally equivalent fragments, anti-C3a antibodies and their functionally equivalent fragments, anti-C3a receptor antibodies and their functionally equivalent fragments, anti-C6 antibodies and their functionally equivalent fragments, anti-C7 antibodies and their functionally equivalent fragments, anti-C8 antibodies and their functionally equivalent fragments, anti-C9 antibodies and their functionally equivalent fragments, anti-properdin antibodies and their functionally equivalent fragments, fusion protein Membrane Cofactor Protein, Decay Accelerating Factor (DAF), C4 bp, Factor H, Factor I, Carboxypeptidase N, vitronectin (S Protein), clusterin, and CD59.

15. The method of claim 11 wherein the complement inhibitor is an antibody or a functionally equivalent fragment thereof.

16. The method of claim 14 wherein the antibody is an anti-Factor D antibody.

17. The method of claim 14 wherein the antibody is selected from the group consisting of anti-properdin antibodies and functionally equivalent fragments thereof, an anti-C5 antibodies and functionally equivalent fragments thereof, and anti-C5a antibodies and functionally equivalent fragments thereof.

18. The method of claim 11 wherein the complement inhibitor inhibits a component of the alternative complement pathway.

19. The method of claim 11 wherein the complement inhibitor is administered to the patient in dosages of from about 3 to 50 milligrams per kilogram of body weight per day.

20. The method of claim 11 wherein the complement inhibitor is administered into the lungs of the patient.
